(19) United States
(12) Patent Application Publication
(10) Pub. No.: US 2026/0157999 A1
(43) Pub. Date: Jun. 11, 2026

(54) DEUTERATED FORMS OF ALKALOID COMPOUNDS AND THERAPEUTIC USES THEREOF

(71) Applicant: Sensorium Therapeutics, Inc., Boston, MA (US)

(72) Inventor: Jacob M. Hooker, Winchester, MA (US)

(21) Appl. No.: 18/705,706

(22) PCT Filed: Oct. 28, 2022
(86) PCT No.: PCT/US2022/048152

§ 371 Date: Apr. 29, 2024

Related U.S. Application Data
(60) Provisional application No. 63/273,692, filed on Oct. 29, 2021.

(51) Int. Cl. A61K 31/404; C07B 59/00; C07D 209/12

(52) U.S. Cl. CPC A61K 31/404; C07B 59/002; C07D 209/12

(57) ABSTRACT

Disclosed are deuterated forms of mesembrine. Also disclosed are pharmaceutical compositions that include deuterated mesembrine.

### RELATED APPLICATIONS

This application claims the benefit of priority to U.S. Provisional Patent Application No. 63/273,692, filed Oct. 29, 2021.

### TECHNICAL FIELD

The present disclosure relates to the field of medicine, including the discovery of alkaloid compounds useful for inhibiting sodium-dependent serotonin transporter (SERT) via oral delivery.

### BACKGROUND

Plants of the genus Sceletium contain indole alkaloids having biological activity useful in treating mental health conditions such as mild to moderate depression. Natural extracts of Sceletium tortuosum, an indigenous herb of South Africa also referred to as “kougoed”, “channa” or “kanna,” can contain the pharmacologically active alkaloids. Mesembrine and mesembrenol are pharmacologically active alkaloids present in Sceletium tortuosum extracts used for treatment of anxiety, stress and mental health conditions.

Natural products obtained from plants of the genus Sceletium contain varying amounts of (−) mesembrine and (+)/(−) mesembrenone. The structure of mesembrine, also known as 3a-(3,4-dimethoxyphenyl)-octahydro-1-methyl-6H-indol-6-one, has been reported by Popelak et al., Naturwiss. 47, 156 (1960), and the configuration by P W Jeffs et al., J. Am. Chem. Soc. 91, 3831 (1969). Naturally occurring (−) mesembrine from Sceletium tortuosum has been reported as having serotonin (5-HT) uptake inhibitory activity useful in treating mental health conditions such as mild to moderate depression. Naturally occurring (+)/(−) mesembrenone from Sceletium tortuosum is reported as a potent selective serotonin reuptake inhibitor (Ki=27 nM).

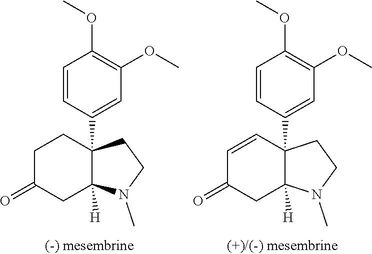 [SMILES: COc1ccc([C@@]23CCC(=O)C[C@@H]2N(C)CC3)cc1OC ; COc1ccc([C@@]23C=CC(=O)C[C@@H]2N(C)CC3)cc1OC]

An analysis of a standardized commercial extract of Sceletium tortuosum was reported in 2011 (obtained as a product under the tradename, Zembrin®) as having 0.35-0.45% total alkaloids, with mesembrenone and mesembrenol comprising ≥60%, and mesembrine contributing <20% (See Harvey et al., “Pharmacological actions of the South African medicinal and functional food plant Sceletium tortuosum and its principal alkaloids,” Journal of Ethnopharmacology 137 (2011) 1124-11292011 and Murbach et. al., “A toxicological safety assessment of a standardized extract of Sceletium tortuosum (Zembrin®) in rats, “Food and Chemical Toxicology 74 (2014) 190-199). The extract gave >80% inhibition at serotonin (5-HT) transporter with potency of the isolated alkaloids at the 5-HT transporter reported as shown in Table A below (Harvey et al., 2011). Referring to the data in Table A, concentration-dependent inhibition was found, with mesembrine being the more active compound (i.e., 20 times more potent than mesembrenone, and 87 times more active than mesembrenol) in the 5-HT transporter assay. A toxicological safety assessment of this standardized extract was subsequently reported in 2014 (Murbach et al., 2014).

TABLE A
Summary of analysis of the concentration response curves of alkaloids
on binding to the 5-HT transporter (Harvey et al., 2011)

5-HT transporter (SERT)

| Compound | Ki (nM) | nH |
|---|---|---|
| Mesembrine | 1.4 | 1.0 |
| Mesembrenone | 27 | 1.0 |

However, bioactive plant extracts for therapeutic consumption can vary widely both seasonally and between different Sceletium tortuosum plants, and fail to provide a sufficiently reproducible and stable phytochemical profile of desired biologically active components. Plants of the genus Sceletium and extracts thereof can vary widely in terms of the total alkaloid content, as well as the chemistry and relative concentrations of individual Sceletium plant derived alkaloids

The therapeutic use of mesembrine and mesembrenone has been limited by the variability and instability of these compounds content in natural extract products and the instability and pharmacokinetic profile of these compounds as obtained from natural products. There remains an unmet need for pharmaceutical compositions comprising higher purity, predictable, stable and reproducible forms of therapeutic alkaloid compounds such as mesembrine and mesembrenone.

### SUMMARY

Described herein are compounds of formula (I) a pharmaceutically acceptable salt thereof:

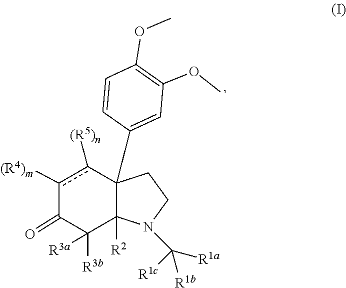

wherein:
   the dashed bond is present or absent;
   each of R^{1a}, R^{1b}, and R^{5c} is independently H or deuterium;
   R² is H or deuterium;
   each of R^{3a} and R^{3b} is independently H or deuterium;
   each R⁴ is independently H or deuterium;
   each R⁵ is independently H or deuterium;
   m is 1 or 2; and
   n is 1 or 2;
   each of m and n is 1 when the dashed bond is present; and
   each of m and n is 2 when the dashed bond is absent;
provided the compound is not:

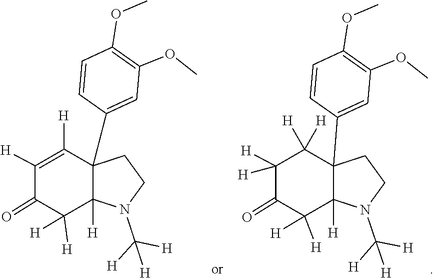 [SMILES: COc1ccc(C23C=CC(=O)CC2N(C)CC3)cc1OC ; COc1ccc(C23CCC(=O)CC2N(C)CC3)cc1OC]

In some embodiments the compound is of formula (Ia) or a pharmaceutically acceptable salt thereof:

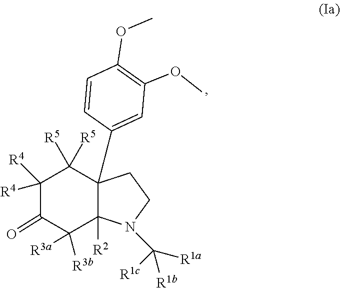

wherein:
   each of R^{1a}, R^{1b}, and R^{1c} is independently H or deuterium;
   R² is H or deuterium;
   each of R^{3a} and R^{3b} is independently H or deuterium;
   each R⁴ is independently H or deuterium; and
   each R⁵ is independently H or deuterium;
provided the compound is not:

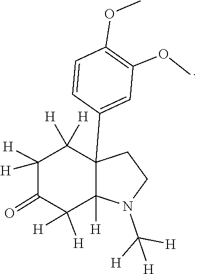 [SMILES: COc1ccc(C23CCC(=O)CC2N(C)CC3)cc1OC]

In some embodiments the compound is of formula (Ib) or a pharmaceutically acceptable salt thereof:

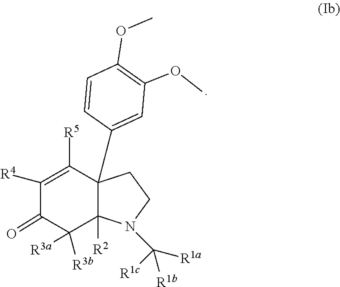

wherein:
   each of R^{1a}, R^{1b}, and R^{1c} is independently H or deuterium;
   R² is H or deuterium;
   each of R^{3a} and R^{3b} is independently H or deuterium;
   R⁴ is independently H or deuterium; and
   R⁵ is independently H or deuterium;
provided the compound is not:

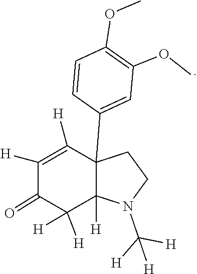 [SMILES: COc1ccc(C23C=CC(=O)CC2N(C)CC3)cc1OC]

In some embodiments the compound is of formula (II) or a pharmaceutically acceptable salt thereof:

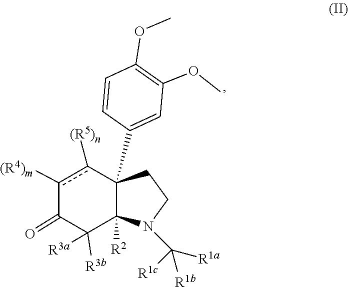

wherein:
   the dashed bond is present or absent;
   each of R^{1a}, R^{1b}, and R^{5c} is independently H or deuterium;
   R² is H or deuterium;
   each of R^{3a} and R^{3b} is independently H or deuterium;
   each R⁴ is independently H or deuterium;
   each R⁵ is independently H or deuterium;
   m is 1 or 2;
   n is 1 or 2;
   each of m and n is 1 when the dashed bond is present; and
   each of m and n is 2 when the dashed bond is absent;
   optionally the compound of formula (II) has the absolute stereochemistry shown;
provided the compound is not:

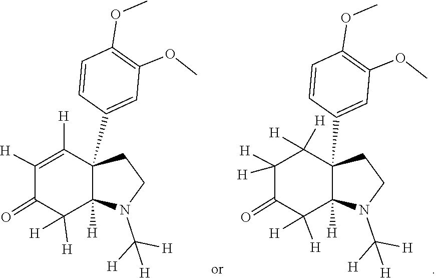 [SMILES: COc1ccc([C@@]23C=CC(=O)C[C@@H]2N(C)CC3)cc1OC ; COc1ccc([C@@]23CCC(=O)C[C@@H]2N(C)CC3)cc1OC]

In some embodiments, the compound is selected from the group consisting of:

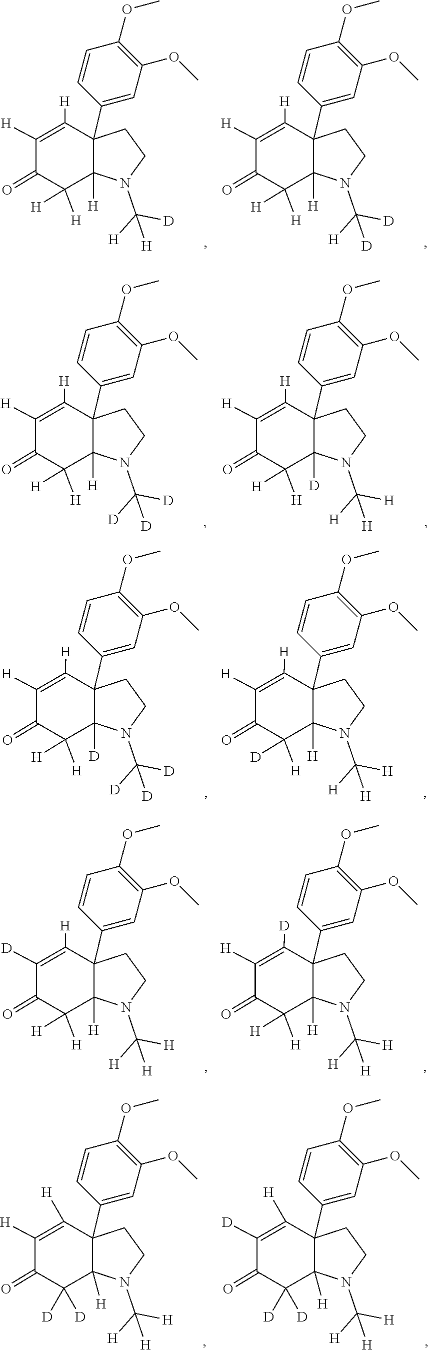

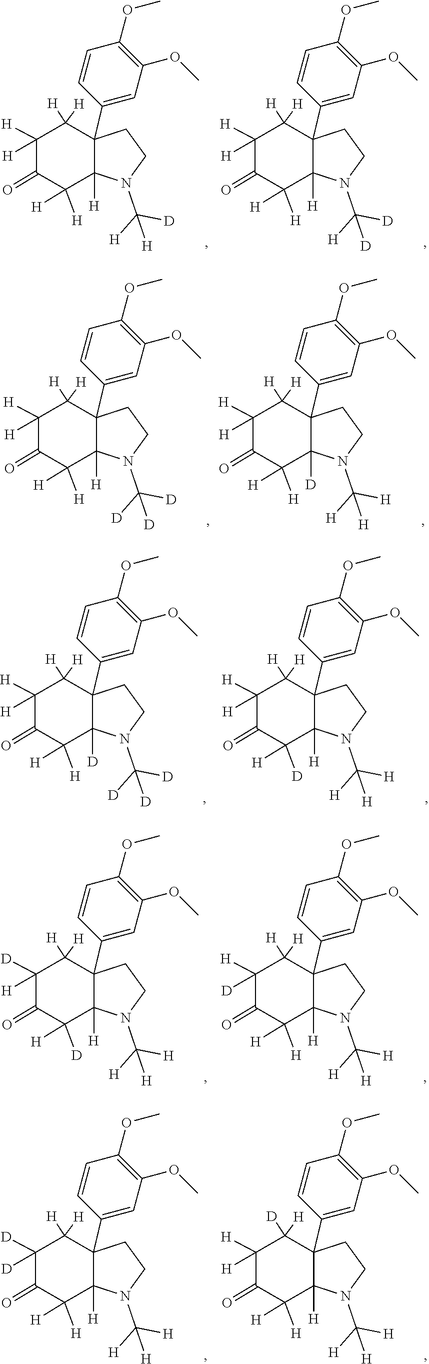

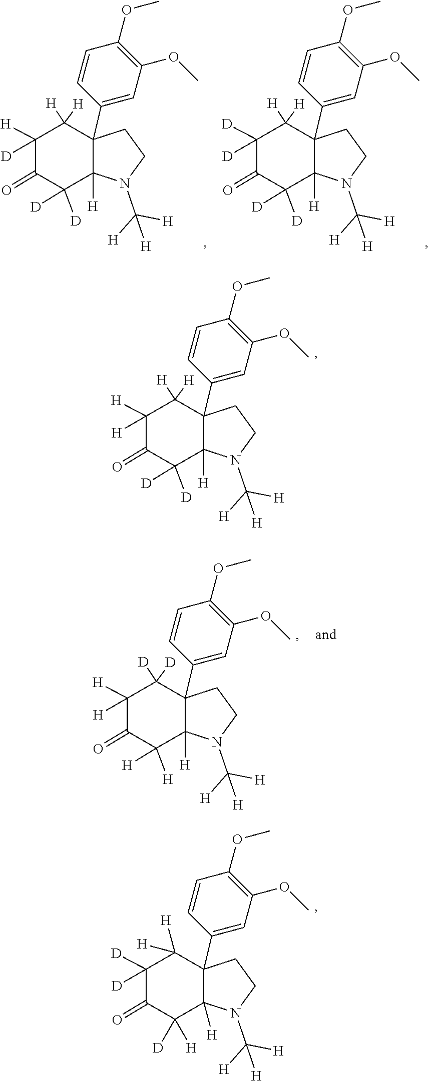

or a pharmaceutically acceptable salt thereof.

In some embodiments, the compound is selected from the group consisting of:

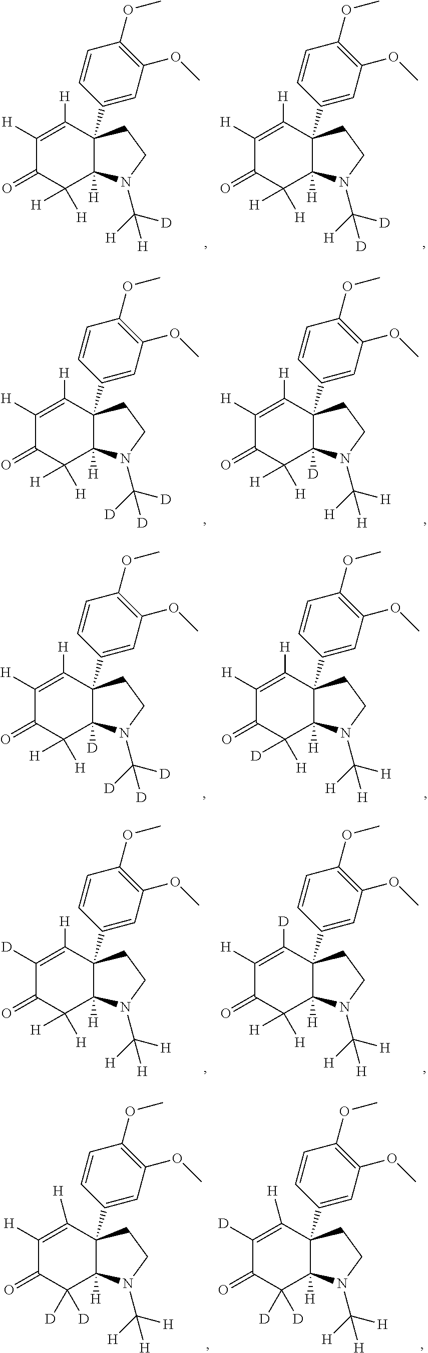

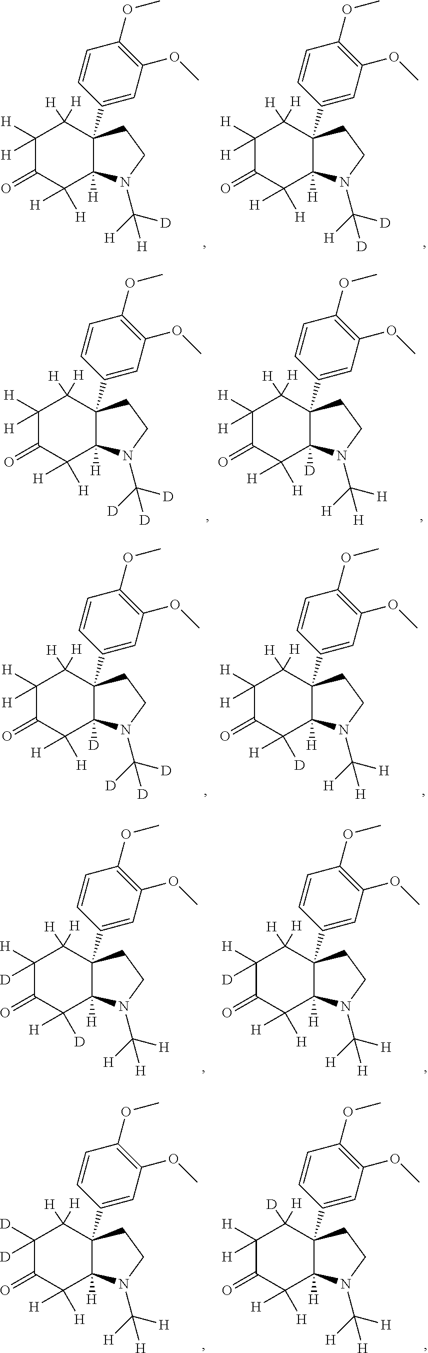

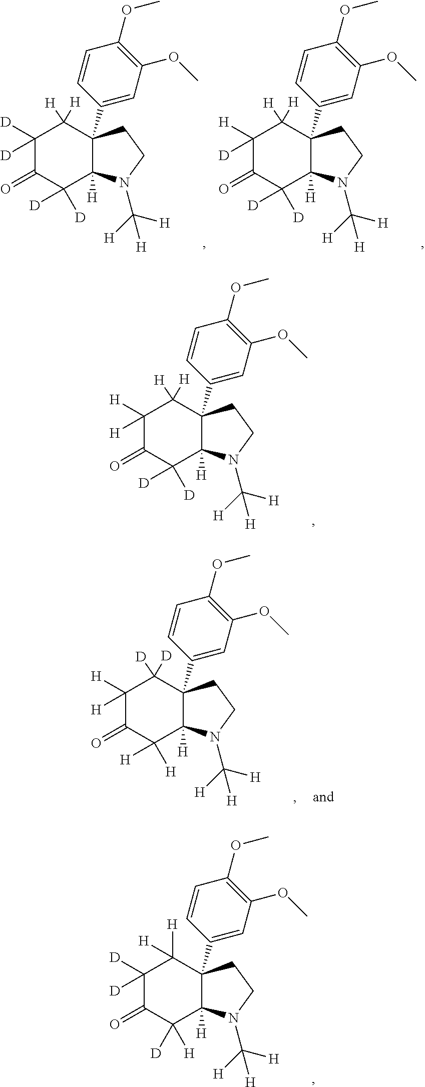

or a pharmaceutically acceptable salt thereof.

In some embodiments, the compound is selected from the group consisting of:

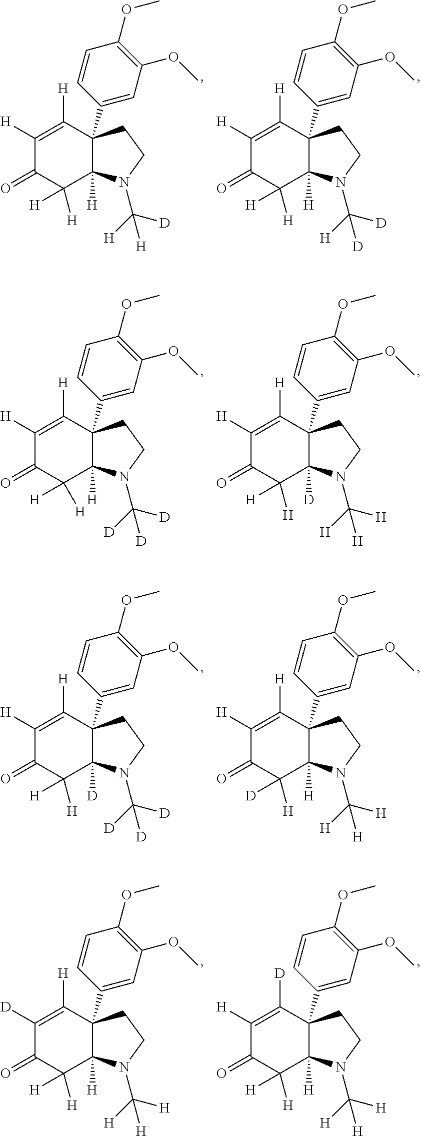

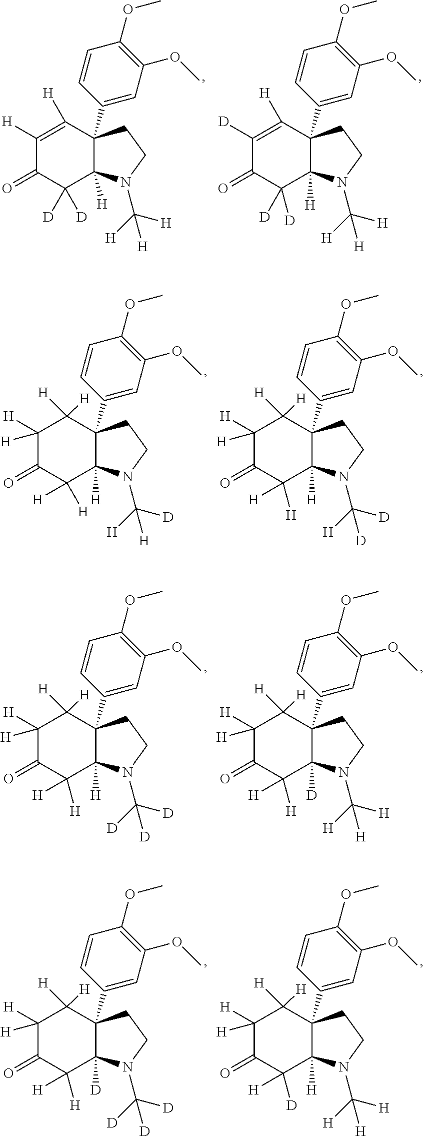

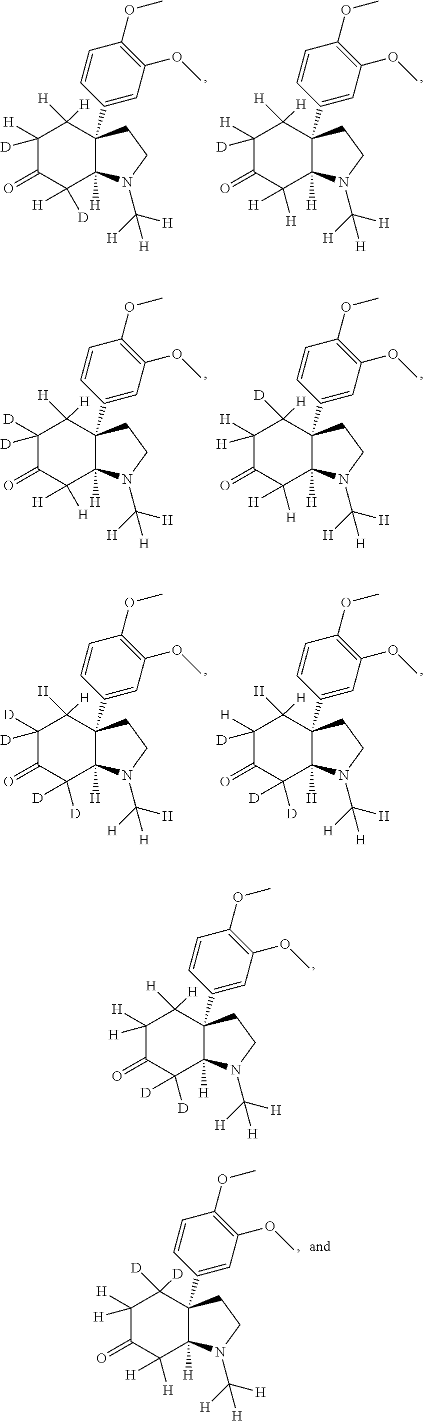

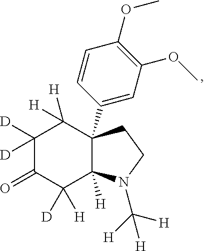 [SMILES: [2H]C1C(=O)C([2H])([2H])C[C@@]2(c3ccc(OC)c(OC)c3)CCN(C)[C@@H]12]

or a pharmaceutically acceptable salt thereof; wherein the compound has the absolute stereochemistry shown.

In certain embodiments, a method of treating a mental health disorder comprises administering to a mammal in need thereof an effective amount of a compound according to the present disclosure.

In certain embodiments, a method of treating an inflammatory condition, comprising administering to a mammal in need thereof an effective amount of a compound according to the present disclosure.

Numerous embodiments are further provided that can be applied to any aspect of the present invention described herein.

### DETAILED DESCRIPTION

The present invention is based, at least in part, on deuterated analogs of mesembrine (e.g., (−) mesembrine) or mesembrenone. Although (−) mesembrine is bioactive with certain desirable pharmacologic effects, certain other properties are less than ideal for use as a therapeutic. For example, the pharmacokinetics described for (−) mesembrine show rapid metabolism and excretion, which an undesirably low half-life in plasma of less than 2 hours. To take advantage of the desirable properties of (−) mesembrine and improve upon absorption, distribution, metabolism and excretion (ADME) that impact pharmacokinetics (PK), compounds have been developed and are described here.

Deuterium substitution is intended in these instances to alter the metabolic fate and or metabolic rate of the natural product. For example, deuterium (single or multiple) alpha to the carbonyl can create a kinetic isotope effect that changes the rate of conversion of mesembrine to mesembranol in vivo during carbonyl reduction, thus altering pharmacokinetics, disposition and pharmacology of the natural product. As another example, deuterium substitution can more generally and empirically alter the metabolic fate of the parent natural product in ways that are beneficial to the ADME/PK profile of the natural product and metabolites. This includes changes to both primary and secondary metabolic pathways.

### Exemplary Compounds of the Invention

In certain embodiments, compounds described have advantageous pharmacokinetic properties compared to (−) mesembrine or mesembrenone.

In some embodiments, a compound according to the present disclosure is of formula (I).

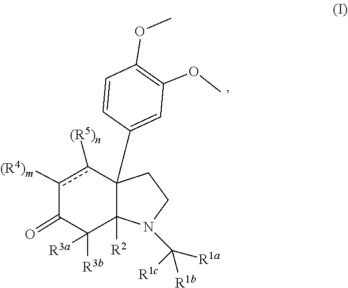

or a pharmaceutically acceptable salt thereof, wherein the dashed bond is present or absent; provided the compound is not:

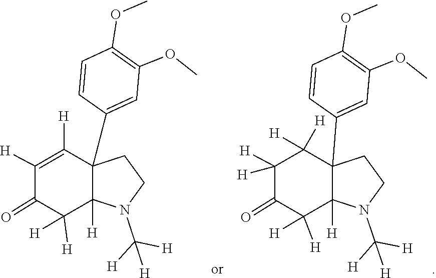 [SMILES: COc1ccc(C23C=CC(=O)CC2N(C)CC3)cc1OC ; COc1ccc(C23CCC(=O)CC2N(C)CC3)cc1OC]

In some embodiments, a compound according to the present disclosure is of formula (Ia):

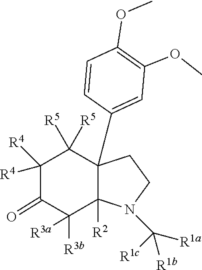

or a pharmaceutically acceptable salt thereof, provided the compound is not:

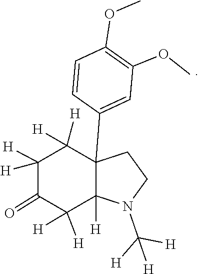 [SMILES: COc1ccc(C23CCC(=O)CC2N(C)CC3)cc1OC]

In some embodiments, a compound according to the present disclosure is of formula (Ib):

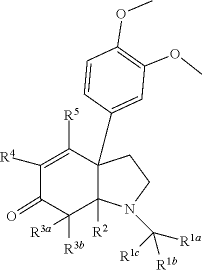

or a pharmaceutically acceptable salt thereof, wherein the dashed bond is present or absent; provided the compound is not:

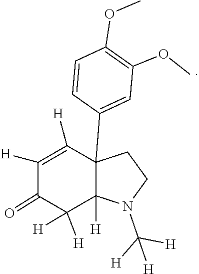 [SMILES: COc1ccc(C23C=CC(=O)CC2N(C)CC3)cc1OC]

In some embodiments, a compound according to the present disclosure is of formula (II):

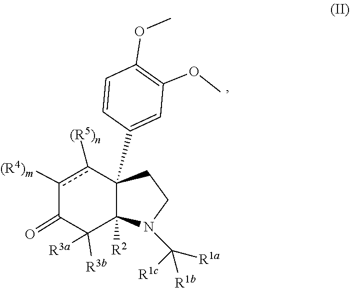

or a pharmaceutically acceptable salt thereof, wherein the dashed bond is present or absent; provided the compound is not:

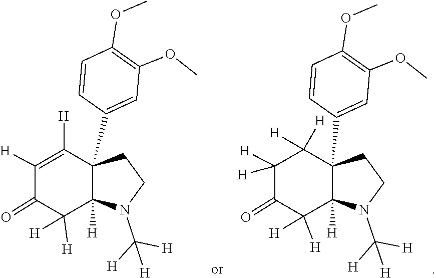 [SMILES: COc1ccc([C@@]23C=CC(=O)C[C@@H]2N(C)CC3)cc1OC ; COc1ccc([C@@]23CCC(=O)C[C@@H]2N(C)CC3)cc1OC]

In some embodiments, each of R^{1a}, R^{1b}, and R^{5c} is independently H or deuterium. In some embodiments, at least one of R^{1a}, R^{1b}, and R^{1c} is deuterium. In some embodiments, at least two of R^{1a}, R^{1b}, and R^{1c} is deuterium. In some embodiments, each of R^{1a}, R^{1b}, and R^{1c} is deuterium.

In some embodiments, R² is H or deuterium. In some embodiments, R² is H. In some embodiments, R² is deuterium.

In some embodiments, each of R^{3a} and R^{3b} is independently H or deuterium. In some embodiments, at least one of R^{3a} and R^{3b} is deuterium. In some embodiments, each of R^{3a} and R^{3b} is deuterium.

In some embodiments, each R⁴ is independently H or deuterium. In some embodiments, at least one R⁴ is deuterium. In some embodiments, m is 2 and both R⁴s are deuterium.

In some embodiments, each R⁵ is independently H or deuterium. In some embodiments, at least one R⁵ is deuterium. In some embodiments, n is 2 and both R⁵s are deuterium.

In some embodiments, m is 1 or 2. In some embodiments, m is 1. In some embodiments, m is 2.

In some embodiments, n is 1 or 2. In some embodiments, n is 1. In some embodiments, n is 2.

In some embodiments, each of m and n is 1 when the dashed bond is present, and each of m and n is 2 when the dashed bond is absent.

In some embodiments, compounds and compositions described herein are used for treatment of anxiety, stress and mental health conditions. In some embodiments, the treatment is of a mammal. In some embodiments, the mammal is a human.

In some embodiments, the compound is selected from the group consisting of:

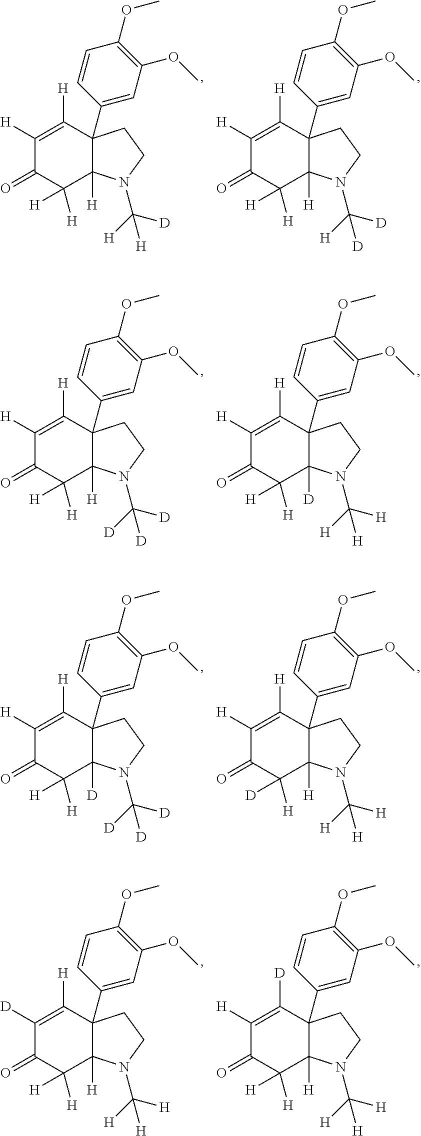

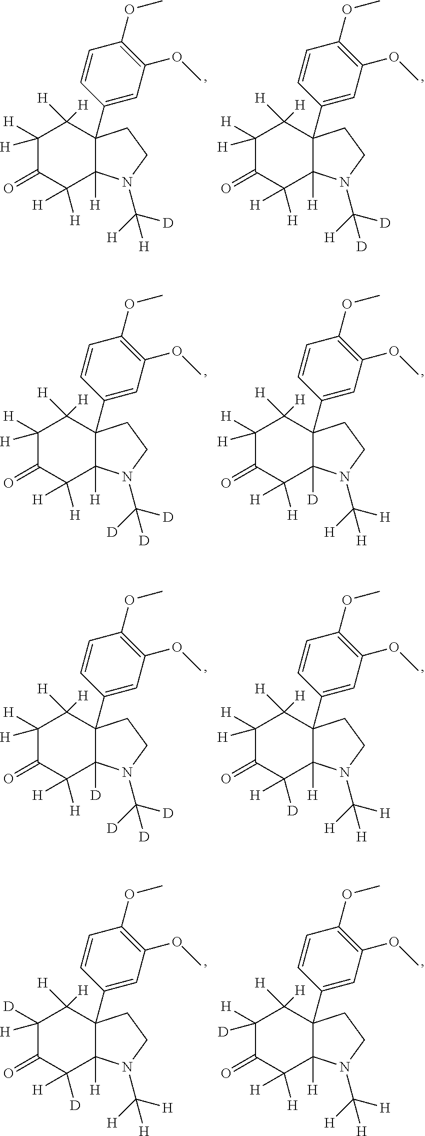

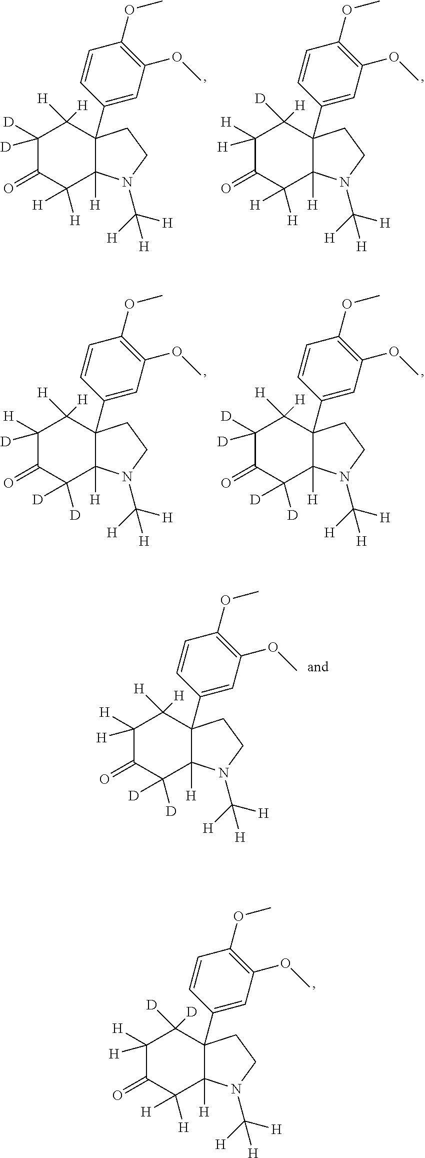 [SMILES: [2H]C1([2H])CC2(c3ccc(OC)c(OC)c3)CCN(C)C2CC1=O ; [2H]C1CC(=O)CC2N(C)CCC12c1ccc(OC)c(OC)c1 ; [2H]C1CC2(c3ccc(OC)c(OC)c3)CCN(C)C2C([2H])([2H])C1=O ; [2H]C1([2H])CC2(c3ccc(OC)c(OC)c3)CCN(C)C2C([2H])([2H])C1=O ; [2H]C1([2H])C(=O)CCC2(c3ccc(OC)c(OC)c3)CCN(C)C12 ; [2H]C1([2H])CC(=O)CC2N(C)CCC21c1ccc(OC)c(OC)c1]

or a pharmaceutically acceptable salt thereof.

In some embodiments, the compound is selected from the group consisting of:

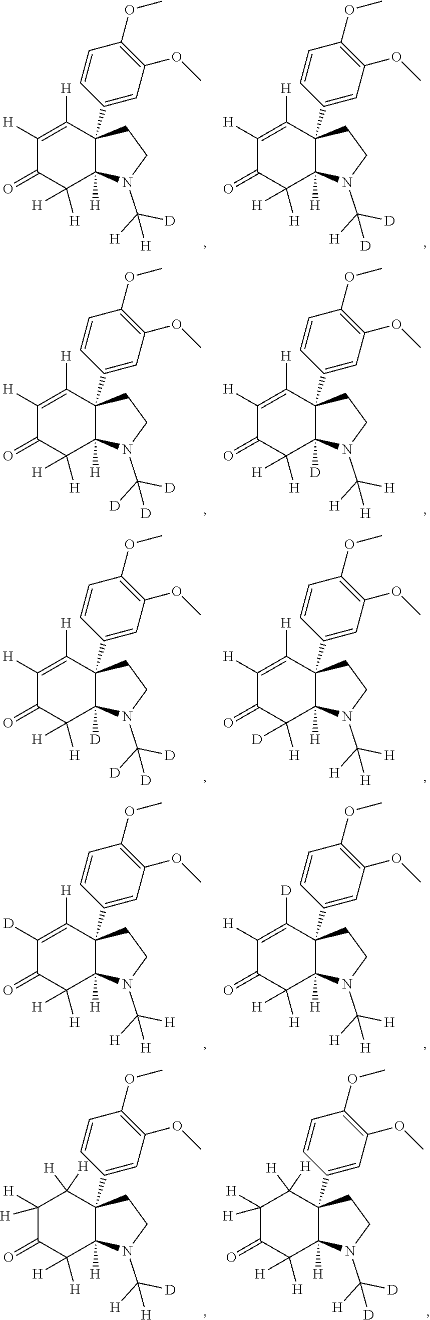

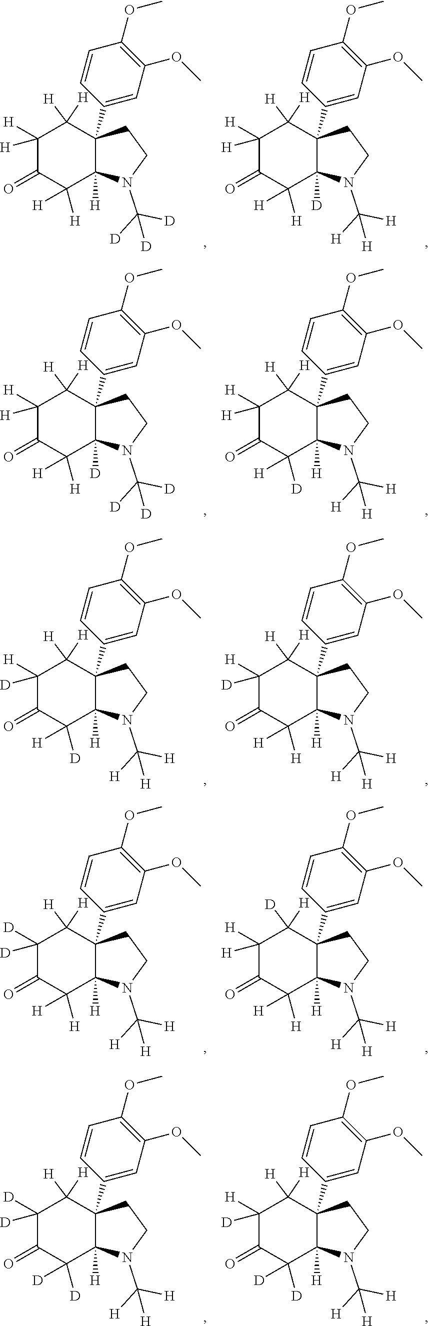

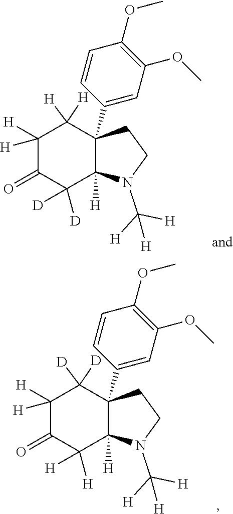

or a pharmaceutically acceptable salt thereof.

In some embodiments, selected from the group consisting of

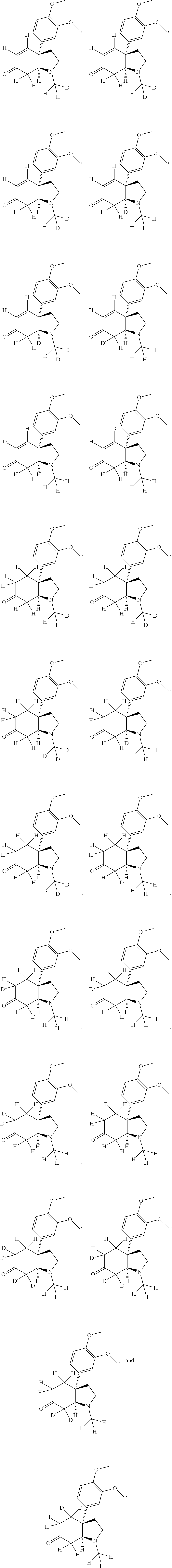

or a pharmaceutically acceptable salt thereof; wherein the compound has the absolute stereochemistry shown.

In certain embodiments, the compound is selected from the group consisting of:

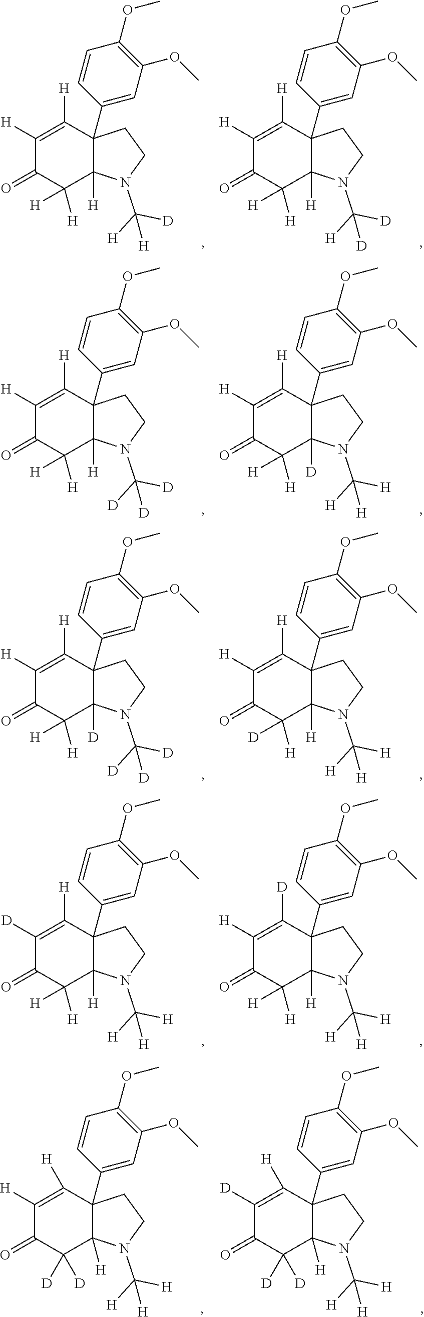

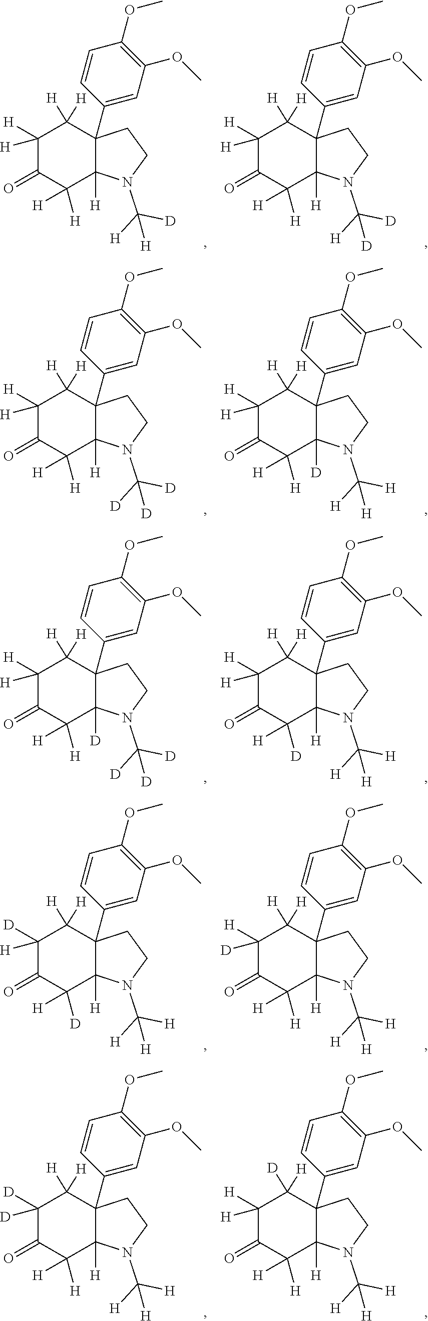

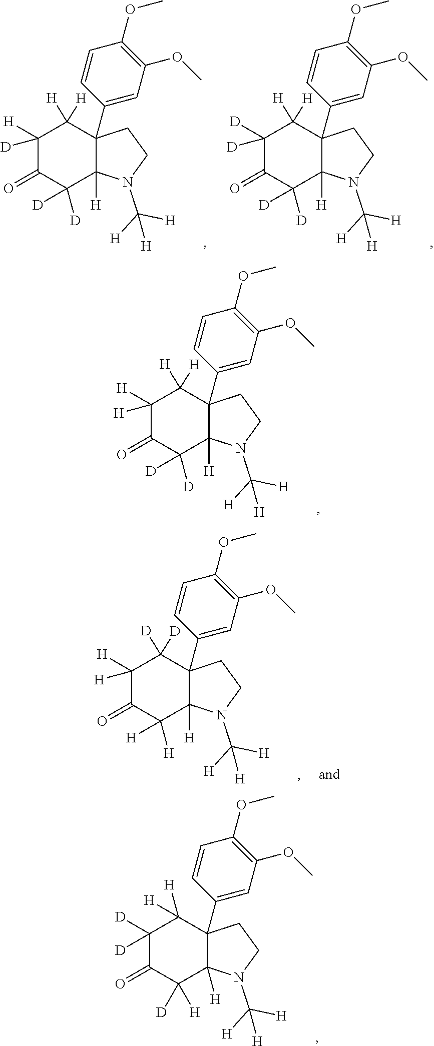

or a pharmaceutically acceptable salt thereof.

In certain embodiments, the compound is selected from the group consisting of:

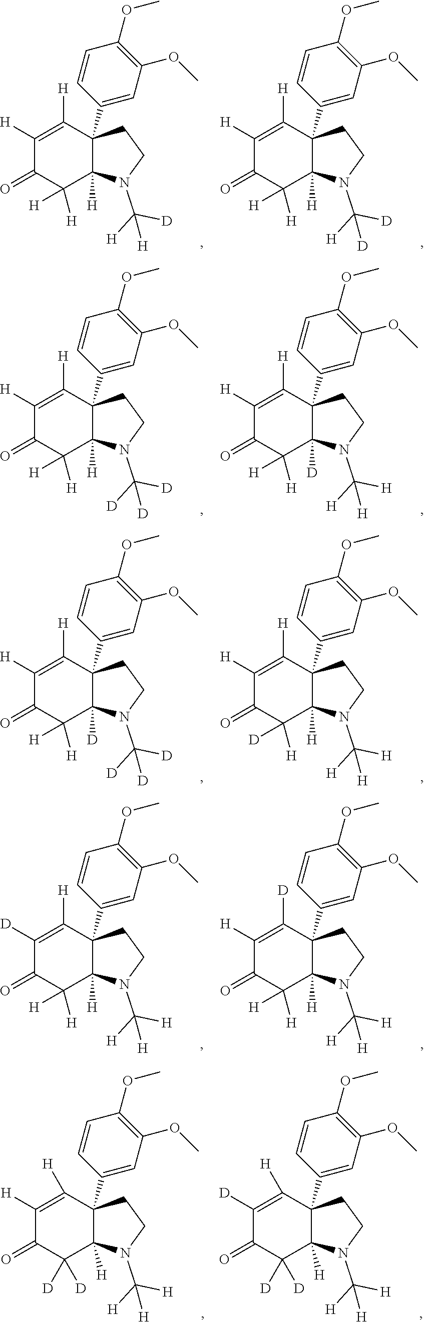

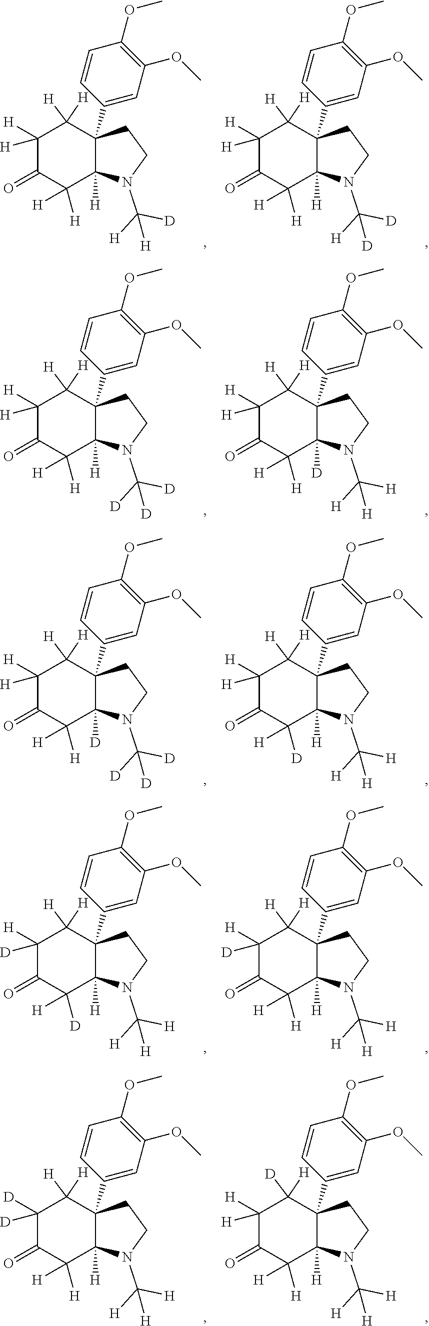

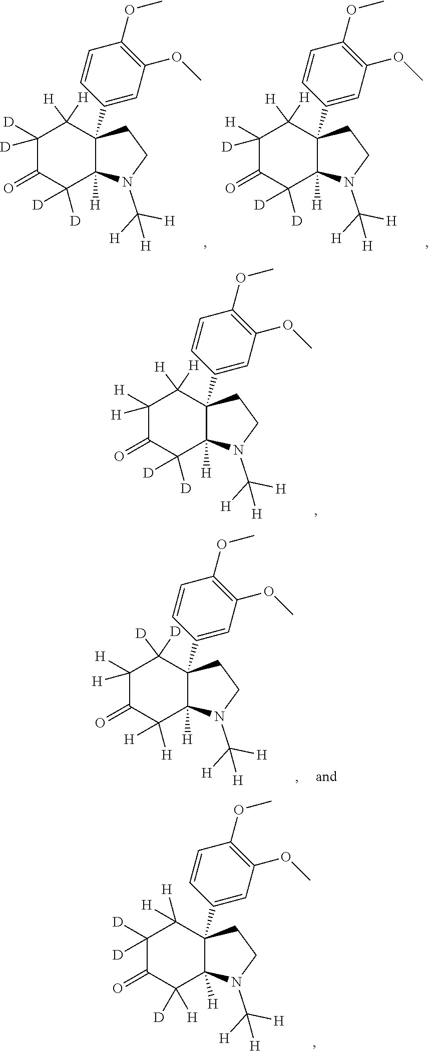

or a pharmaceutically acceptable salt thereof.

In certain embodiments, the compound is selected from the group consisting of:

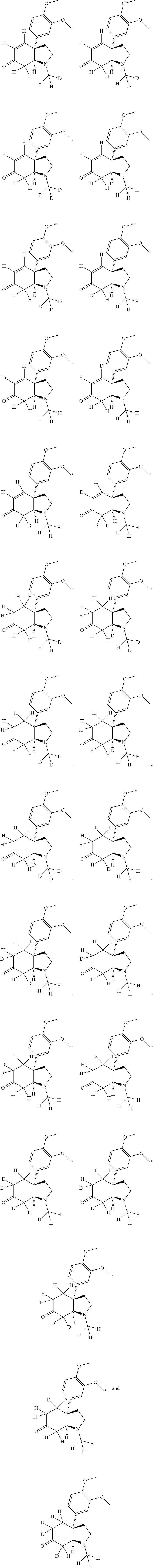

or a pharmaceutically acceptable salt thereof; wherein the compound has the absolute stereochemistry shown.

### Pharmaceutical Compositions and Methods of Treatment

In certain embodiments, the present application is directed to a pharmaceutical composition comprising an active pharmaceutical ingredient. In certain embodiments, the pharmaceutical composition comprises a compound as disclosed herein as the active pharmaceutical ingredient (API) and a pharmaceutically acceptable carrier comprising one or more excipients. In some embodiments, the pharmaceutical composition optionally further comprises an additional therapeutic compound (i.e., agent) with the pharmaceutically acceptable carrier. The pharmaceutical composition can be a medicament.

Pharmaceutically acceptable carriers include those known in the art. The choice of a pharmaceutically acceptable carrier can depend, for example, on the desired route of administration of the composition. A pharmaceutical composition (preparation) can be administered to a subject by any of a number of routes of administration including, for example, parenteral administration (e.g. intravenously, subcutaneously, or intramuscularly), oral administration (for example, tablets, and capsules); absorption through the oral mucosa (e.g., sublingually) or transdermally (for example as a patch applied to the skin) or topically (for example, as a cream, ointment or spray applied to the skin).

In some embodiments, pharmaceutical compositions comprising compounds of Formula (I) or pharmaceutically acceptable salts thereof can be formulated for oral administration. For example, a compound provided herein can be combined with suitable compendial excipients to form an oral unit dosage form, such as a capsule or tablet, containing a target dose of a compound of Formula (I). The drug product can be prepared by first manufacturing the compound of Formula (I) as an active pharmaceutical ingredient (API), followed by roller compaction/milling with intragranular excipients and blending with extra granular excipients. A Drug Product can contain the selected compound of Formula (I) as the API and excipient components in a tablet in a desired dosage strength of Compound 1. The blended material can be compressed to form tablets and then film coated. The excipients can be selected from materials appropriate for inclusion in a pharmaceutical composition for an intended purpose and route of delivery including providing a desired manufacturing and stability properties and/or desired in vivo characteristics or other properties to the pharmaceutical composition. In some embodiments, the pharmaceutical composition can include a compound of Formula (I) as the API in combination with a filler (e.g., a form of microcrystalline cellulose), a dry binder or disintegrant (e.g., a cross-linked polymer), a glidant (e.g., colloidal silicon dioxide) and/or a lubricant (e.g., magnesium stearate). In some embodiments, the pharmaceutical composition can comprise a material such as an extended release or disintegrant involved in carrying or transporting the API pharmaceutical agent from one organ, or portion of the body, to another organ, or portion of the body of a subject, including materials to desirable control the absorption of the API in the intestine.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

To prepare solid dosage forms for oral administration, the active ingredient is mixed with one or more pharmaceutically acceptable carriers, such as sodium citrate or dicalcium phosphate, and/or any of the following: (1) fillers or extenders, (2) binders, (3) humectants, (4) disintegrating agents, (5) solution retarding agents, (6) absorption accelerators, (7) wetting agents, (8) absorbents, (9) lubricants, (10) complexing agents, and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using suitable excipients. The pharmaceutical compositions according to the present invention may contain conventional pharmaceutical carriers and/or auxiliary agents. In some embodiments, he pharmaceutical compositions according to the present invention may contain conventional carrier agents including a binder, a lubricant and/or a glidant selected from those products and materials generally used in pharmaceutical industry for preparation of pharmaceutical compositions for an intended route of administration.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

Liquid dosage forms useful for oral administration include pharmaceutically acceptable carriers and the active ingredient provided as a solid form for reconstitution prior to administration or as a liquid (e.g., solutions, suspensions, or emulsions). In addition to the active ingredient, a liquid dosage forms may contain inert diluents commonly used in the art. For example, formulations of pharmaceutically acceptable compositions for injection can include aqueous solutions such as water or physiologically buffered saline or other solvents or vehicles suitable for the intended route of administration. In some embodiments, the pharmaceutical composition is formulated for parenteral administration.

The therapeutically effective amount of a pharmaceutical composition can be determined by human clinical trials to determine the safe and effective dose for a patient with a relevant diagnosis. It is generally understood that the effective amount of the compound may vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the pharmaceutical composition at a dose and dose interval determined to be safe and effective for the patient.

The present disclosure includes the use of pharmaceutically acceptable salts of compounds of the invention in the compositions and methods of the present invention. Pharmaceutically-acceptable salts include, for example, acid-addition salts and base-addition salts. The acid that is added to a compound to form an acid-addition salt can be an organic acid or an inorganic acid. A base that is added to a compound to form a base-addition salt can be an organic base or an inorganic base. In some embodiments, a pharmaceutically-acceptable salt is a metal salt, in some embodiments, a pharmaceutically-acceptable salt is an ammonium salt. For example, a pharmaceutically acceptable acid addition salt can exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

In some embodiments, methods of treating a patient suffering from a disease comprise administering to a patient a composition comprising a compound disclosed herein for the treatment or prevention of a mental health disorder. In some embodiments, methods of treating a patient suffering from a disease comprise administering to a patient a composition comprising a compound disclosed herein for the treatment or prevention of a diagnosed condition selected from anxiety and depression. In some embodiments, the compound disclosed herein is administered to the patient in a unit dose. In some embodiments, a method comprises the administration to a patient in need thereof of a therapeutically effective amount of a compound of Formula (I) for the treatment of a disease selected from the group consisting of mild to moderate depression and major depressive episodes. In some embodiments, a method comprises the administration to a patient in need thereof of a therapeutically effective amount of a compound of Formula (I) for the treatment of anxiety. In some embodiments, a method comprises the administration to a patient in need thereof of a therapeutically effective amount of a compound of Formula (I) for the treatment of depression. In some embodiments, a method comprises the administration to a patient in need thereof of a therapeutically effective amount of a compound of Formula (I) for the treatment of a condition selected from the group consisting of: anxiety associated with depression, anxiety with depression, mixed anxiety and depressive disorder. In some embodiments, a method comprises the administration to a patient in need thereof of a therapeutically effective amount of a compound of Formula (I) for the treatment of anxiety and hysteria or anxiety and depression.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e., not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

The term “modulate” as used herein includes the inhibition or suppression of a function or activity (such as cell proliferation) as well as the enhancement of a function or activity.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” or “salt” is used herein to refer to an acid addition salt or a basic addition salt which is suitable for or compatible with the treatment of patients.

The term “pharmaceutically acceptable acid addition salt” as used herein means any non-toxic organic or inorganic salt of any base compounds represented by Formula I. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds of Formula I are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds of Formula I for laboratory use, or for subsequent conversion to a pharmaceutically acceptable acid addition salt.

The term “pharmaceutically acceptable basic addition salt” as used herein means any non-toxic organic or inorganic base addition salt of any acid compounds represented by Formula I or any of their intermediates. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

Many of the compounds useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers).

Furthermore, certain compounds which contain alkenyl groups may exist as Z (zusammen) or E (entgegen) isomers. In each instance, the disclosure includes both mixture and separate individual isomers.

Some of the compounds may also exist in tautomeric forms. Such forms, although not explicitly indicated in the formulae described herein, are intended to be included within the scope of the present disclosure.

“Prodrug” or “pharmaceutically acceptable prodrug” refers to a compound that is metabolized, for example hydrolyzed or oxidized, in the host after administration to form the compound of the present disclosure (e.g., compounds of formula I). Typical examples of prodrugs include compounds that have biologically labile or cleavable (protecting) groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. Examples of prodrugs include using ester or phosphoramidate as biologically labile or cleavable (protecting) groups. The prodrugs of this disclosure are metabolized to produce a compound of Formula I. The present disclosure includes within its scope, prodrugs of the compounds described herein. Conventional procedures for the selection and preparation of suitable prodrugs are described, for example, in “Design of Prodrugs” Ed. H. Bundgaard, Elsevier, 1985.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

The phrases “parenteral administration” and “administered parenterally” as used herein means modes of administration other than enteral and topical administration, usually by injection, and includes, without limitation, intravenous, intraocular (such as intravitreal), intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

The term “Log of solubility”, “Log S” or “log S” as used herein is used in the art to quantify the aqueous solubility of a compound. The aqueous solubility of a compound significantly affects its absorption and distribution characteristics. A low solubility often goes along with a poor absorption. Log S value is a unit stripped logarithm (base 10) of the solubility measured in mol/liter.

### EXAMPLES

Table of Abbreviations

| | |
|---|---|
| Ac | Acetyl |
| Et | Ethyl |
| Me | Methyl |
| Ms | Methanesulfonyl |
| Ts | p-Toluenesulfonyl |
| Tol | toluene |
| HPLC | High Performance Liquid Chromatography |
| DMSO | Dimethyl sulfoxide |
| LC-MS | Liquid chromatography - mass spectrometry |
| NMR | Nuclear magnetic resonance |
| m | multiplet |
| d | Doublet |
| s | singlet |
| t | triplet |
| SFC | Supercritical fluid chromatography |
| IPA | isopropanol |
| ESI | Electrospray ionization |
| EtOAC | Ethyl acetate |
| t-buOK | Potassium tertbutoxide |
| ACN | acetonitrile |
| Ac | acetate |
| MS | Molecular sieves |
| DCM | dichloromethane |
| TMS | trimethylsilyl |

### Example 1: Synthesis of rac-(3aS,7aR)-3a-(3,4-dimethoxyphenyl)-1-methyl-1,2,3,3a,7,7a-hexahydro-6H-indol-6-one-7,7-d₂ and rac-(3aS,7aR)-3a-(3,4-dimethoxyphenyl)-1-methyl-1,2,3,3a, 7,7a-hexahydro-6H-indol-6-one-7-d (Deuterated Analogs of Mesembrenone)

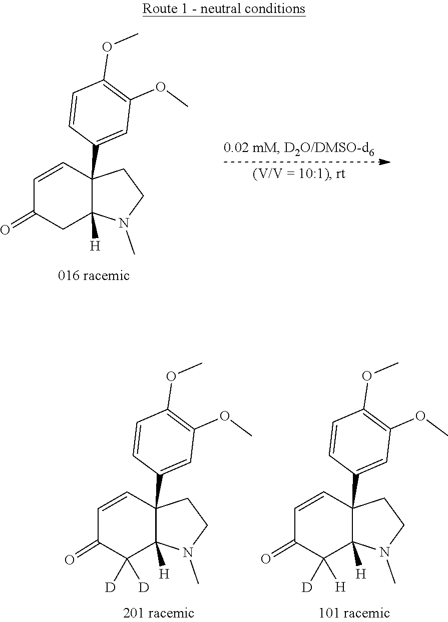 [SMILES: COc1ccc([C@]23C=CC(=O)C[C@H]2N(C)CC3)cc1OC ; [2H]C1([2H])C(=O)C=C[C@]2(c3ccc(OC)c(OC)c3)CCN(C)[C@H]12 ; [2H]C1C(=O)C=C[C@]2(c3ccc(OC)c(OC)c3)CCN(C)[C@H]12]

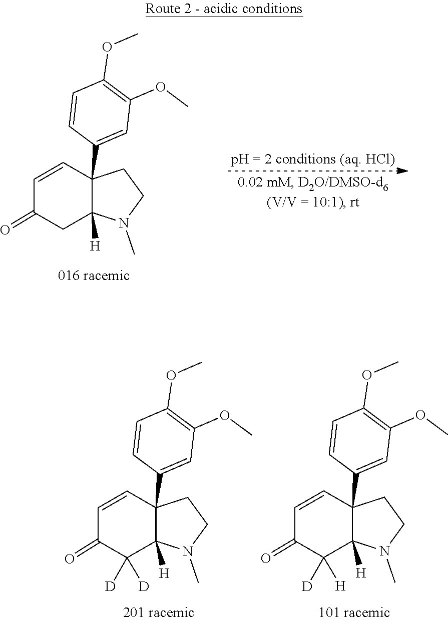 [SMILES: COc1ccc([C@]23C=CC(=O)C[C@H]2N(C)CC3)cc1OC ; [2H]C1([2H])C(=O)C=C[C@]2(c3ccc(OC)c(OC)c3)CCN(C)[C@H]12 ; [2H]C1C(=O)C=C[C@]2(c3ccc(OC)c(OC)c3)CCN(C)[C@H]12]

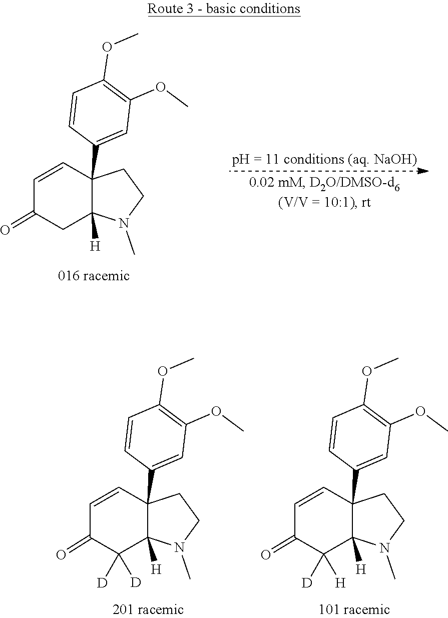 [SMILES: COc1ccc([C@]23C=CC(=O)C[C@H]2N(C)CC3)cc1OC ; [2H]C1([2H])C(=O)C=C[C@]2(c3ccc(OC)c(OC)c3)CCN(C)[C@H]12 ; [2H]C1C(=O)C=C[C@]2(c3ccc(OC)c(OC)c3)CCN(C)[C@H]12]

### Example 2: Synthesis of 3a-(3,4-dimethoxyphenyl)-1-methyloctahydro-6H-indol-6-one-5,7-d₂, (3aR,7aR)-3a-(3,4-dimethoxyphenyl)-1-methyloctahydro-6H-indol-6-one-7,7-d₂, 3a-(3,4-dimethoxyphenyl)-1-methyloctahydro-6H-indol-6-one-5,7,7-d₃, and (3aR,7aR)-3a-(3,4-dimethoxyphenyl)-1-methyloctahydro-6H-indol-6-one-5,5,7,7-d₄ (Deuterated Analogs of Mesembrine)

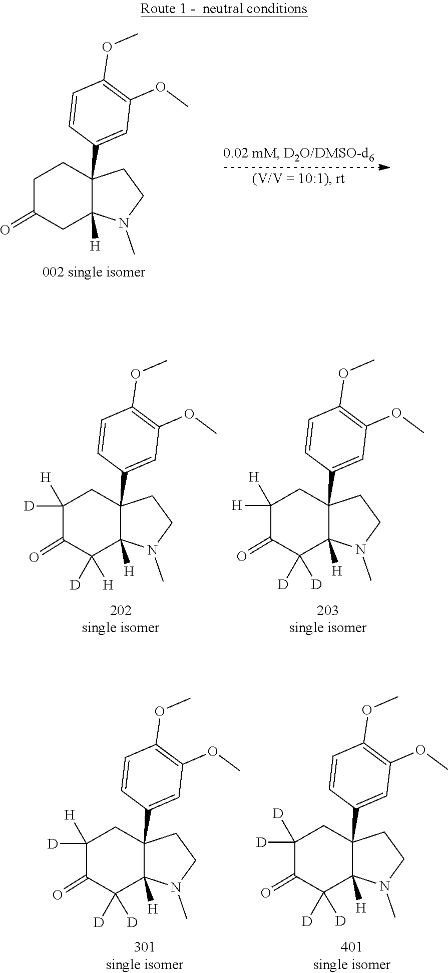 [SMILES: COc1ccc([C@]23CCC(=O)C[C@H]2N(C)CC3)cc1OC ; [2H]C1C[C@]2(c3ccc(OC)c(OC)c3)CCN(C)[C@@H]2C([2H])C1=O ; [2H]C1([2H])C(=O)CC[C@]2(c3ccc(OC)c(OC)c3)CCN(C)[C@H]12 ; [2H]C1C[C@]2(c3ccc(OC)c(OC)c3)CCN(C)[C@@H]2C([2H])([2H])C1=O ; [2H]C1([2H])C[C@]2(c3ccc(OC)c(OC)c3)CCN(C)[C@@H]2C([2H])([2H])C1=O]

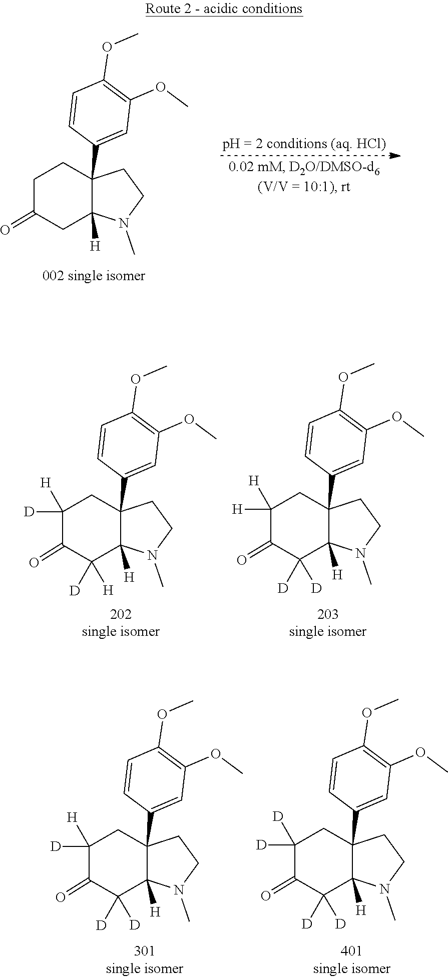 [SMILES: COc1ccc([C@]23CCC(=O)C[C@H]2N(C)CC3)cc1OC ; [2H]C1C[C@]2(c3ccc(OC)c(OC)c3)CCN(C)[C@@H]2C([2H])C1=O ; [2H]C1([2H])C(=O)CC[C@]2(c3ccc(OC)c(OC)c3)CCN(C)[C@H]12 ; [2H]C1C[C@]2(c3ccc(OC)c(OC)c3)CCN(C)[C@@H]2C([2H])([2H])C1=O ; [2H]C1([2H])C[C@]2(c3ccc(OC)c(OC)c3)CCN(C)[C@@H]2C([2H])([2H])C1=O]

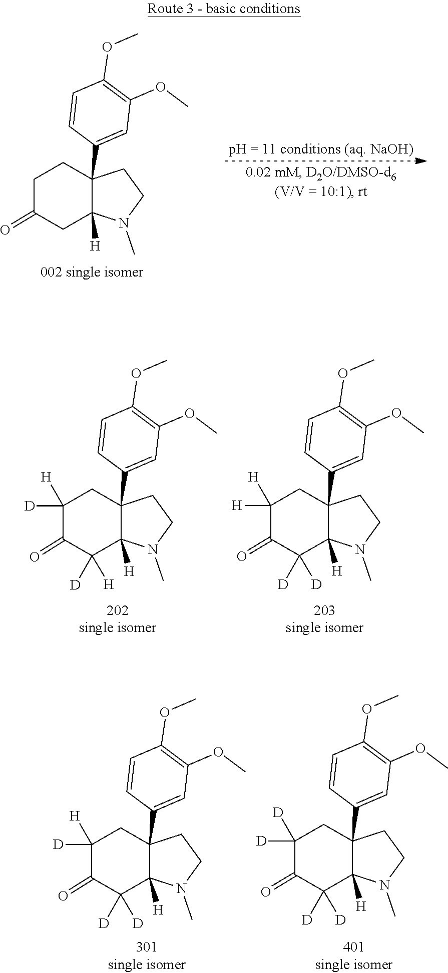 [SMILES: COc1ccc([C@]23CCC(=O)C[C@H]2N(C)CC3)cc1OC ; [2H]C1C[C@]2(c3ccc(OC)c(OC)c3)CCN(C)[C@@H]2C([2H])C1=O ; [2H]C1([2H])C(=O)CC[C@]2(c3ccc(OC)c(OC)c3)CCN(C)[C@H]12 ; [2H]C1C[C@]2(c3ccc(OC)c(OC)c3)CCN(C)[C@@H]2C([2H])([2H])C1=O ; [2H]C1([2H])C[C@]2(c3ccc(OC)c(OC)c3)CCN(C)[C@@H]2C([2H])([2H])C1=O]

### Example 2A: Synthesis of 3a-(3,4-dimethoxyphenyl)-1-methyloctahydro-6H-indol-6-one-5,7-d₂, (3aS,7aS)-3a-(3,4-dimethoxyphenyl)-1-methyloctahydro-6H-indol-6-one-7,7-d₂, 3a-(3,4-dimethoxyphenyl)-1-methyloctahydro-6H-indol-6-one-5,7,7-d₃, and (3aS,7aS)-3a-(3,4-dimethoxyphenyl)-1-methyloctahydro-6H-indol-6-one-5,5,7,7-d₄ (Deuterated Analogs of Mesembrine)

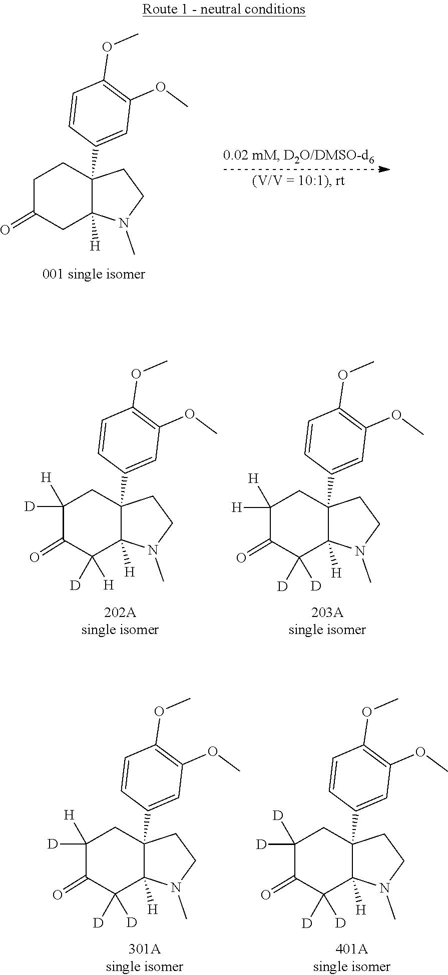 [SMILES: COc1ccc([C@@]23CCC(=O)C[C@@H]2N(C)CC3)cc1OC ; [2H]C1C[C@@]2(c3ccc(OC)c(OC)c3)CCN(C)[C@H]2C([2H])C1=O ; [2H]C1([2H])C(=O)CC[C@@]2(c3ccc(OC)c(OC)c3)CCN(C)[C@@H]12 ; [2H]C1C[C@@]2(c3ccc(OC)c(OC)c3)CCN(C)[C@H]2C([2H])([2H])C1=O ; [2H]C1([2H])C[C@@]2(c3ccc(OC)c(OC)c3)CCN(C)[C@H]2C([2H])([2H])C1=O]

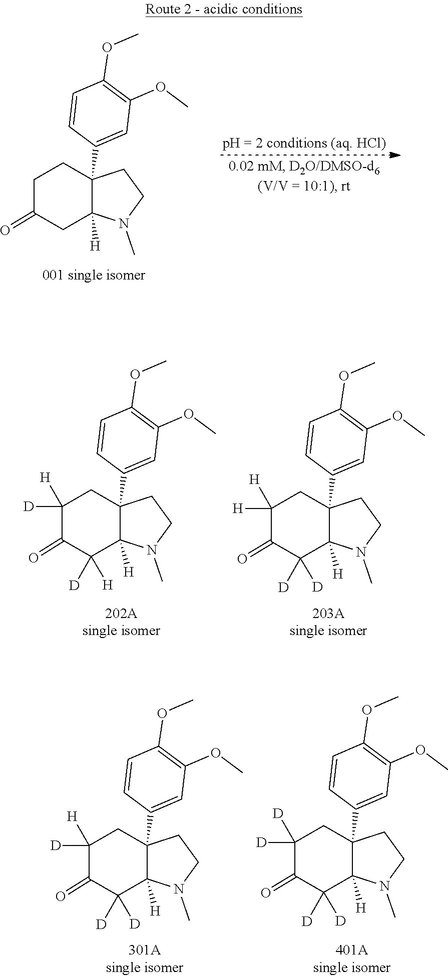 [SMILES: COc1ccc([C@@]23CCC(=O)C[C@@H]2N(C)CC3)cc1OC ; [2H]C1C[C@@]2(c3ccc(OC)c(OC)c3)CCN(C)[C@H]2C([2H])C1=O ; [2H]C1([2H])C(=O)CC[C@@]2(c3ccc(OC)c(OC)c3)CCN(C)[C@@H]12 ; [2H]C1C[C@@]2(c3ccc(OC)c(OC)c3)CCN(C)[C@H]2C([2H])([2H])C1=O ; [2H]C1([2H])C[C@@]2(c3ccc(OC)c(OC)c3)CCN(C)[C@H]2C([2H])([2H])C1=O]

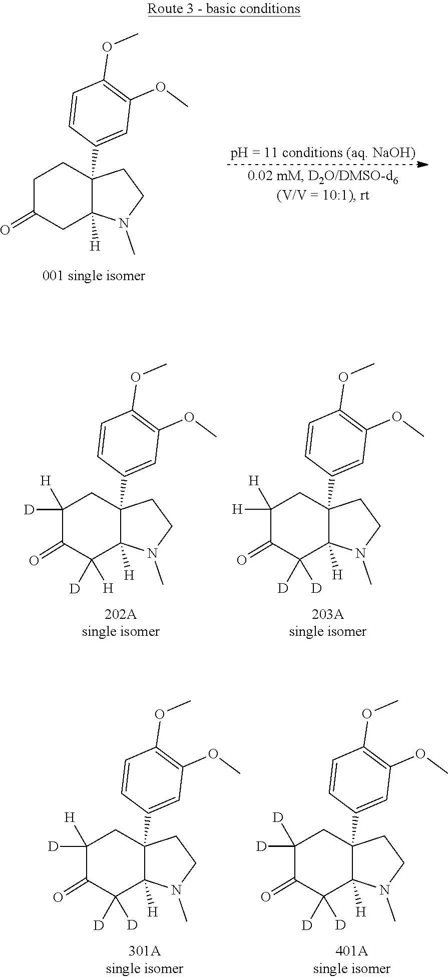 [SMILES: COc1ccc([C@@]23CCC(=O)C[C@@H]2N(C)CC3)cc1OC ; [2H]C1C[C@@]2(c3ccc(OC)c(OC)c3)CCN(C)[C@H]2C([2H])C1=O ; [2H]C1([2H])C(=O)CC[C@@]2(c3ccc(OC)c(OC)c3)CCN(C)[C@@H]12 ; [2H]C1C[C@@]2(c3ccc(OC)c(OC)c3)CCN(C)[C@H]2C([2H])([2H])C1=O ; [2H]C1([2H])C[C@@]2(c3ccc(OC)c(OC)c3)CCN(C)[C@H]2C([2H])([2H])C1=O]

### Example 3: Synthesis of rac-(3aS,7aR)-3a-(3,4-dimethoxyphenyl)-1-methyl-1,2,3,3a,7,7a-hexahydro-6H-indol-6-one-7a-d and rac-3a-(3,4-dimethoxyphenyl)-1-methyloctahydro-6H-indol-6-one-7a-d (Deuterated Analogs of Mesembrenone and Mesembrine)

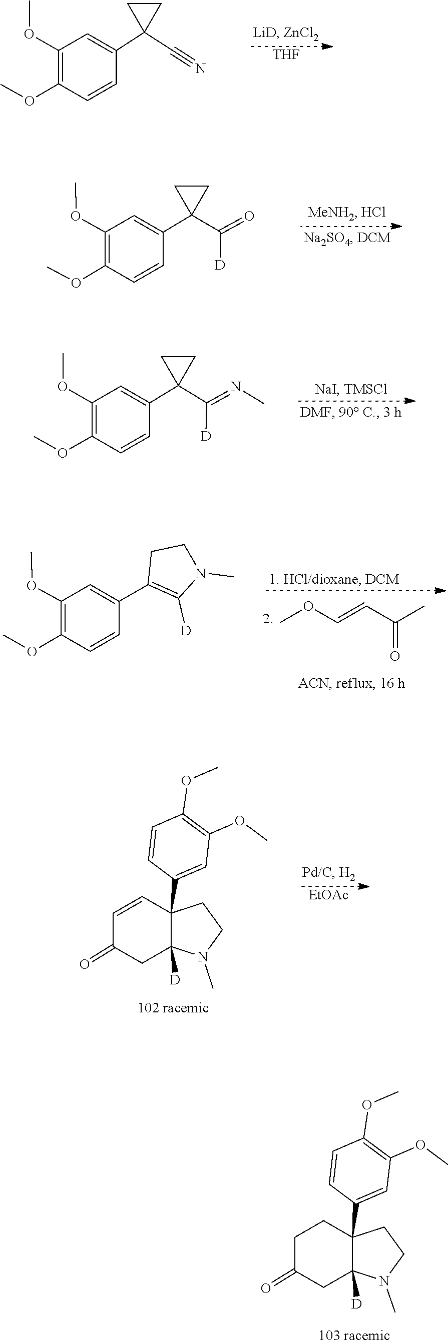

### Example 4: Synthesis of rac-3a-(3,4-dimethoxyphenyl)-1-(methyl-d3)-1,2,3,3a,7,7a-hexahydro-6H-indol-6-one and rac-3a-(3,4-dimethoxyphenyl)-1-(methyl-d3)octahydro-6H-indol-6-one (Deuterated Mesembrenone and Mesembrine and Analogs)

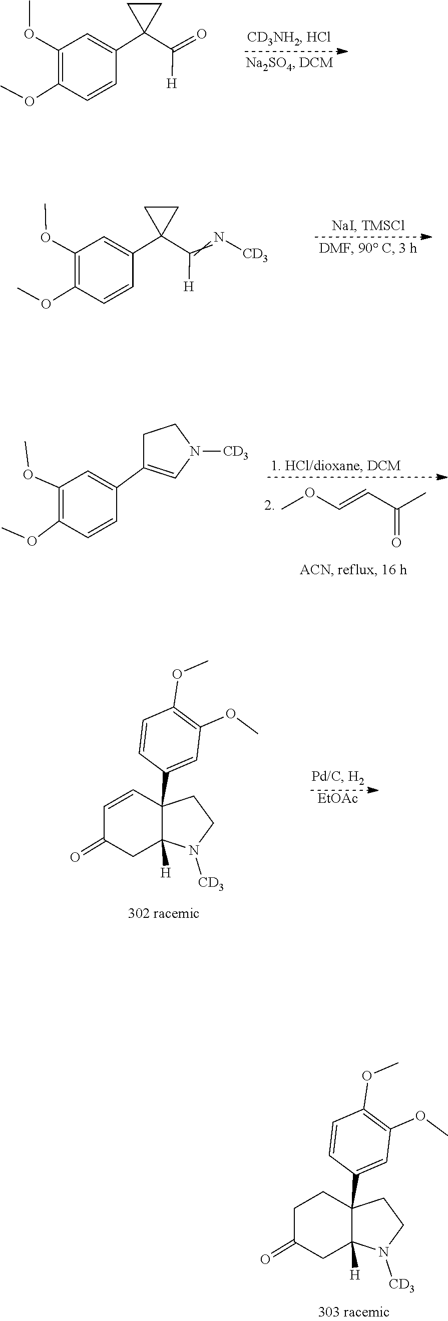

### Example 5: Synthesis of (3aS,7aS)-3a-(3,4-dimethoxyphenyl)-1-methyloctahydro-6H-indol-6-one-5,5,7,7-d₄ (402)

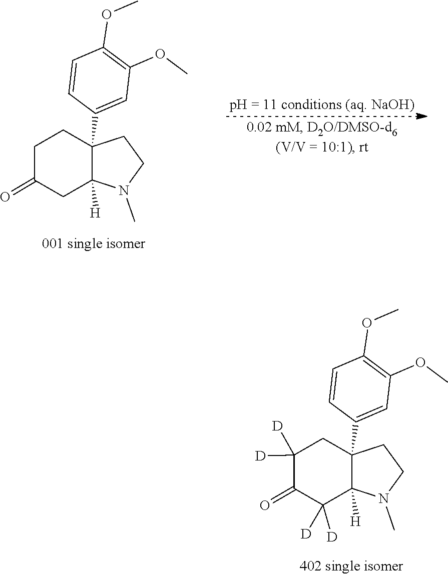 [SMILES: COc1ccc([C@@]23CCC(=O)C[C@@H]2N(C)CC3)cc1OC ; [2H]C1([2H])C[C@@]2(c3ccc(OC)c(OC)c3)CCN(C)[C@H]2C([2H])([2H])C1=O]

A solution of 001 (100 mg, 345 umol) in D₂O (1.0 mL) and CD₃CN (0.1 mL) was allowed to stir at 25° C. for 2 hours. To the reaction mixture was added NaOH in D₂O until the solution had pH=11. The reaction mixture was filtered using a C18 column and lyophilized to give 402 (49 mg) as a colorless oil. LC-MS (ESI⁺) m/z 294.1 (M+H)⁺ ¹H NMR (400 MHz, DMSO-d₆) δ 6.98-6.94 (m, 1H), 7.00-6.94 (m, 1H), 6.92 (s, 1H), 3.76 (d, J=16.0 Hz, 5H), 3.05-2.99 (m, 1H), 2.84 (s, 1H), 2.53 (d, J=2.0 Hz, 1H), 2.25 (s, 5H), 2.11-1.93 (m, 3H).

### Example 6: Synthesis of (3aR,7aS)-3a-(3,4-dimethoxyphenyl)-1-methyl-1,2,3,3a,7,7a-hexahydro-6H-indol-6-one-5,7,7-d₃ (304)

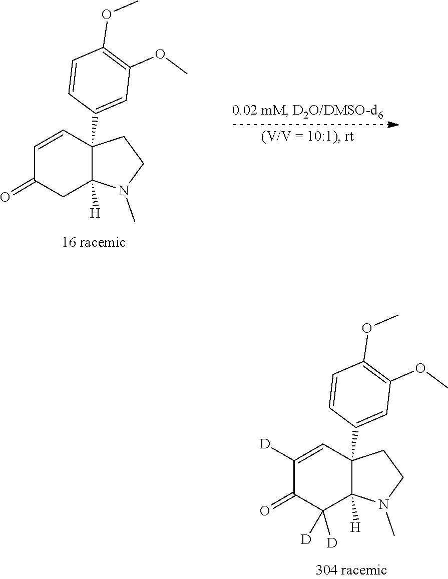 [SMILES: COc1ccc([C@@]23C=CC(=O)C[C@@H]2N(C)CC3)cc1OC ; [2H]C1=C[C@@]2(c3ccc(OC)c(OC)c3)CCN(C)[C@H]2C([2H])([2H])C1=O]

A mixture of 016 (100. mg, 348 umol) in D₂O (0.5 mL) and DMSO-d₆ (1 g, 12.80 mmol, 1.00 mL) was degassed and purged with N₂ 3 times, and then the mixture was allowed to stir at 60° C. for 168 h. The mixture was lyophilized to give 304 (72.6 mg, 92% purity) as a yellow solid. LC-MS (ESI⁺) m/z 291.1 (M+H)⁺. ¹H NMR (400 MHz, DMSO-d6) δ=7.10-6.74 (m, 4H), 3.79-3.76 (m, 6H), 3.13 (dt, J=2.8, 8.6 Hz, 1H), 2.72-2.62 (m, 1H), 2.49-2.42 (m, 1H), 2.35-2.35 (m, 1H), 2.29-2.16 (m, 3H), 1.91-1.89 (m, 1H).

### Example B1: SERT Inhibition Assay

SERT inhibition was measured using a Neruotransmitter Transportation Fluorescence assay. Briefly, stable 5HHH cells were prepared in a 384 microwell plate. Compounds were prepared by in assay buffer (20 mM HEPES, 0.1% BSA). The compounds were added to the plated cells and were incubated for 30 minutes at 37° C. 25 μL of dye solution (Molecular Devices Neurotransmitter Transporter Uptake Assay Kit) was added per well and incubated for 30 minutes at 37° C. The plates were then read on a plate reader.

The in vitro SERT inhibition was measured for the compounds described herein. The results are provided as follows: A: IC₅₀</=50 nM or lower; B: 50 nM<IC₅₀</=100 nM; C: 100 nM<IC₅₀</=500 nM; D: 500 nM<IC50</=1 micromolar; E: IC50 >1 micromolar.

The in vitro SERT inhibition was measured for the compounds listed in the table below.

| Compound | SERT IC₅₀ (nM) |
|---|---|
| 304 | C |
| 402 | A |

1-30. (canceled)

31. A compound of formula (I) or a pharmaceutically acceptable salt thereof: 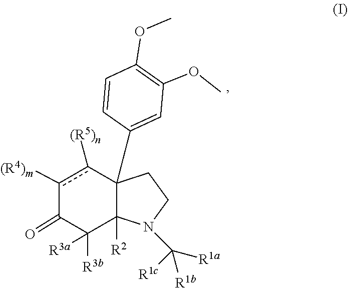
wherein:
the dashed bond is absent or present;
each of R^{1a}, R^{1b}, and R⁵, is independently H or deuterium;
R² is H or deuterium;
each of R^{3a} and R^{3b} is independently deuterium or H;
each R⁴ is independently deuterium or H;
each R⁵ is independently H or deuterium;
m is 1 or 2; and
n is 1 or 2;
each of m and n is 2 when the dashed bond is absent; and each of m and n is 1 when the dashed bond is present;
provided that at least one of R^{1a}, R^{1b}, R^{1c}, R², R^{3a}, R^{3b} and R⁵ is deuterium when R⁴ is hydrogen, n is 2, and m is 2; and provided the compound is not

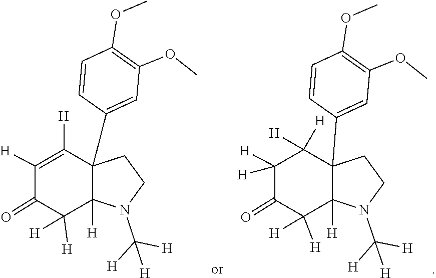 [SMILES: COc1ccc(C23C=CC(=O)CC2N(C)CC3)cc1OC ; COc1ccc(C23CCC(=O)CC2N(C)CC3)cc1OC]

32. The compound of claim 31, wherein the compound is a compound of formula (Ia) or a compound of formula (Ib), or a pharmaceutically acceptable salt thereof: 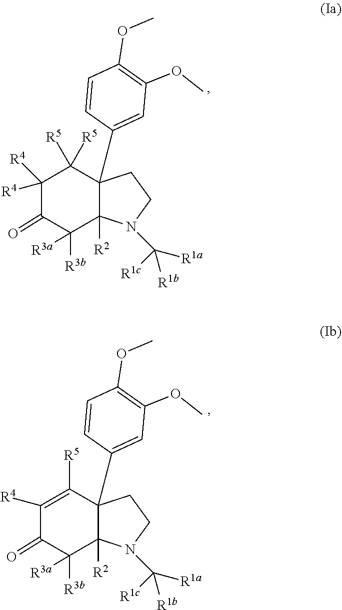
wherein:
each of R^{1a}, R^{1b}, and R^{1c} is independently H or deuterium;
R² is H or deuterium;
each of R^{3a} and R^{3b} is independently H or deuterium;
each R⁴ is independently H or deuterium; and
each R⁵ is independently H or deuterium;
provided the compound is not:

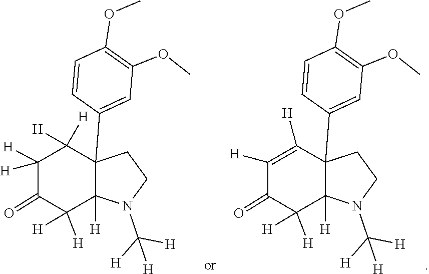 [SMILES: COc1ccc(C23CCC(=O)CC2N(C)CC3)cc1OC ; COc1ccc(C23C=CC(=O)CC2N(C)CC3)cc1OC]

33. The compound of claim 32, wherein R² is H.

34. The compound of claim 33, wherein each of R^{1a}, R^{1b}, and R^{1c} is independently deuterium.

35. The compound of claim 34, wherein each of R^{3a}, R^{3b}, R⁴ and R⁵ is H.

36. The compound of claim 33, wherein each of R^{1a}, R^{1b}, and R^{1c} is independently H.

37. The compound of claim 36, wherein each R⁵ is H.

38. The compound of claim 37, wherein R^{3a} is deuterium.

39. The compound of claim 38, wherein R^{3b} is H.

40. The compound of claim 38, wherein R^{3b} is deuterium.

41. The compound of claim 40, wherein the compound is a compound of formula (Ia), or a pharmaceutically acceptable salt thereof, and each R⁴ is deuterium.

42. The compound of claim 40, wherein the compound is a compound of formula (Ib), or a pharmaceutically acceptable salt thereof, and R⁴ is deuterium.

43. The compound of claim 31, wherein at least one of R^{1a}, R^{1b}, and R^{1c} is deuterium.

44. The compound of claim 31, wherein at least one of R^{3a}, R^{3b}, R⁴ and R⁵ is deuterium.

45. The compound of claim 32, wherein the compound is a compound of formula (Ia).

46. The compound of claim 32, wherein the compound is a compound of formula (Ib).

47. A compound of formula (II) or a pharmaceutically acceptable salt thereof: 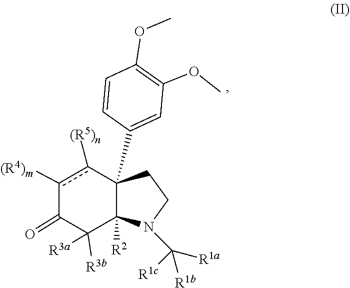
wherein:
the dashed bond is present or absent;
each of R^{1a}, R^{1b}, and R⁵, is independently H or deuterium;
R² is H or deuterium;
each of R^{3a} and R^{3b} is independently H or deuterium;
each R⁴ is independently H or deuterium;
each R⁵ is independently H or deuterium;
m is 1 or 2;
n is 1 or 2;
each of m and n is 1 when the dashed bond is present; and
each of m and n is 2 when the dashed bond is absent;
optionally the compound has the absolute stereochemistry shown; and
wherein:
a. R² is deuterium and each of R^{1a}, R^{1b}, R^{1c}, R^{3a}, R^{3b}, R⁴ and R⁵ is independently H;
b. at least one of R^{3a}, R^{3b}, R⁴ and R⁵ is deuterium and R^{1a}, R^{1b}, R^{1c} and R² are each H; or
c. at least one of R^{1a}, R^{1b}, and R^{1c} is deuterium and each of R², R^{3a}, R^{3b}, R⁴ and R⁵ is H.

48. The compound of claim 31, selected from the group consisting of: 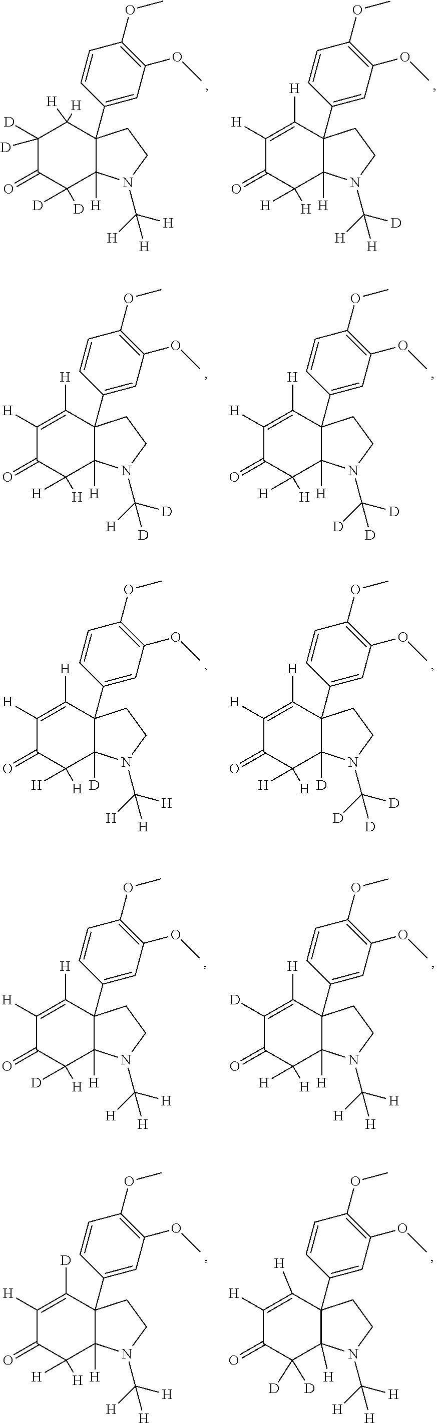 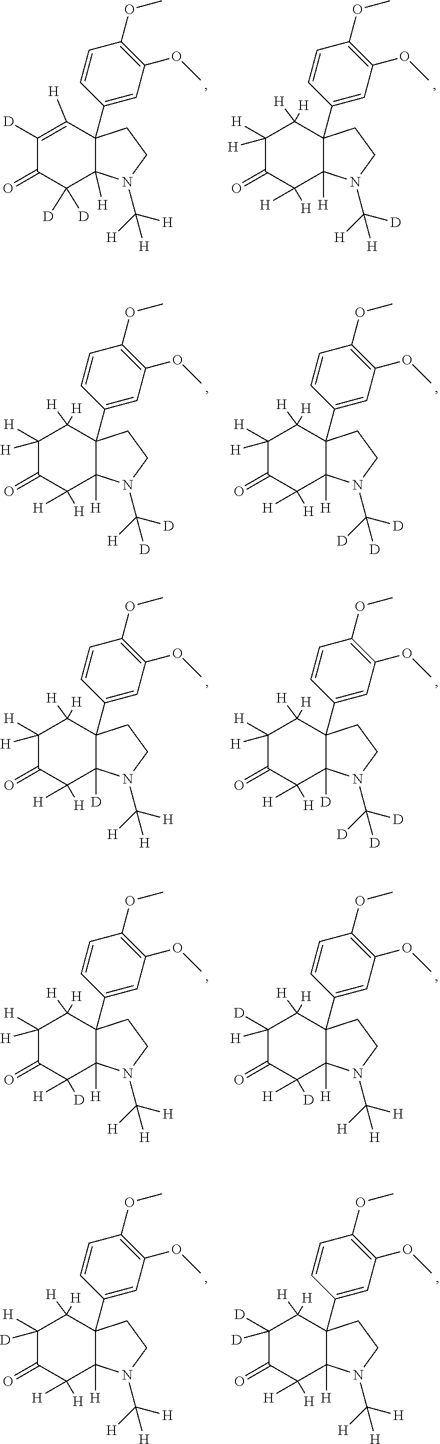 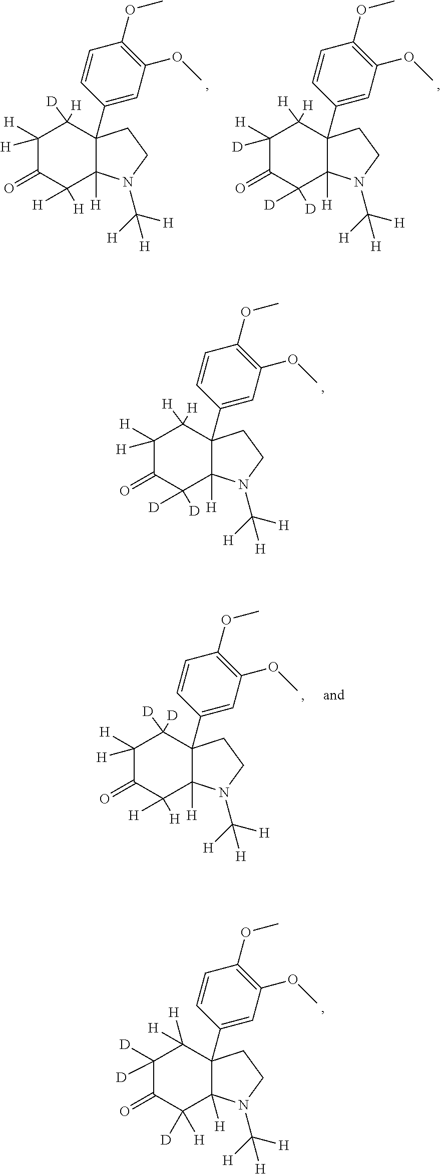 [SMILES: [2H]C1CC(=O)CC2N(C)CCC12c1ccc(OC)c(OC)c1 ; [2H]C1CC2(c3ccc(OC)c(OC)c3)CCN(C)C2C([2H])([2H])C1=O ; [2H]C1([2H])C(=O)CCC2(c3ccc(OC)c(OC)c3)CCN(C)C12 ; [2H]C1([2H])CC(=O)CC2N(C)CCC21c1ccc(OC)c(OC)c1 ; [2H]C1C(=O)C([2H])([2H])CC2(c3ccc(OC)c(OC)c3)CCN(C)C12]
or a pharmaceutically acceptable salt thereof.

49. The compound of claim 31, wherein the compound is 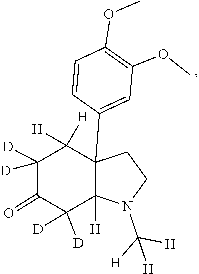 [SMILES: [2H]C1([2H])CC2(c3ccc(OC)c(OC)c3)CCN(C)C2C([2H])([2H])C1=O]
or a pharmaceutically acceptable salt thereof.

50. A method of treating anxiety, stress or depression in a mammal, the method comprising administering to a mammal in need thereof an effective amount of a compound according to claim 31.
